(19) United States
(12) Patent Application Publication
(10) Pub. No.: US 2017/0158599 A1
(43) Pub. Date: Jun. 8, 2017

(54) COMPOSITIONS AND METHODS COMPRISING SALICYLATES AND POLYSALICYLATES

(71) Applicants: Joseph D. CECCOLI, Farmingville, NY (US); Michael L. INGRASSIA, Hauppauge, NY (US); Christopher M. JUDD, Riverhead, NY (US); Brian R. COSTELLO, Port Jefferson Station, NY (US); Biocogent, LLC, Stony Brook, NY (US)

(72) Inventors: Joseph D. Ceccoli, Farmingville, NY (US); Brian R. Costello, Port Jefferson Station, NY (US); Michael L. Ingrassia, Hauppauge, NY (US); Christopher M. Judd, Riverhead, NY (US)

(21) Appl. No.: 15/327,294

(22) PCT Filed: Jul. 17, 2015
(86) PCT No.: PCT/US15/40854

§ 371 Date: Jan. 18, 2017

Related U.S. Application Data
(60) Provisional application No. 62/026,154, filed on Jul. 18, 2014.

(51) Int. Cl. C07C 67/08; A61K 8/37; C08G 63/81; A61Q 19/00; C08G 63/06; A61K 31/765; A61K 8/85

(52) U.S. Cl. CPC C07C 67/08; A61K 31/765; A61K 8/37; A61K 8/85; A61Q 19/00; C08G 63/065; C08G 63/81; A61K 2800/74

(57) ABSTRACT

The present technology relates to synthesis of compositions comprising salicylates, polysalicylates and other derivatives of salicylic acid, and personal care and pharmaceutical compositions comprising the same.

### BACKGROUND

The present technology relates to synthesis of compositions comprising salicylates and polysalicylates, and personal care (including cosmetic) and pharmaceutical (including veterinary) compositions comprising the same.

Salicylic acid (2-hydroxybenzoic acid) is naturally present in a variety of plant tissues, and in particular is present at high levels in several popular fruits, vegetables and spices. One of the richest natural sources of salicylic acid (SA) is willow bark, which has a history of traditional use as an analgesic and antipyretic. These anti-inflammatory effects of SA mainly result from its effects on cyclooxygenase activities, which decrease synthesis of inflammatory prostaglandins and increase production of various pro-resolving lipid mediators that help to decrease inflammation.

Salicylic acid as an oral anti-inflammatory drug is mainly consumed as acetylsalicylic acid (aspirin). Salicylic acid also is used as an active ingredient in topical formulations to treat skin disorders such as psoriasis, warts and, most notably, acne vulgaris. SA also is included in personal care (including cosmetic) formulations to improve the smoothness of skin by promoting exfoliation.

In general, topical efficacy of SA results mostly from its keratolytic and comedolytic activities and, for certain indications, its bacteriostatic properties also are important.

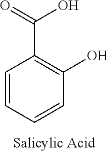

There is always interest in novel active ingredients that are useful for pharmaceutical (including veterinary) and personal care (including cosmetic) applications. Such ingredients can be created as combinations of known molecules one or more of which has beneficial effect on the skin. These compounds often have the character of a pro-ingredient which, upon hydrolysis in the skin, release their active component, which might be protected from degradation or have enhanced availability (e.g., skin penetration), in the pro-ingredient form. An example of this type of ingredient is tetra-isopalmitoyl ascorbic acid which is designed to enhance both the stability of vitamin C and its delivery into the skin. The ability of such a pro-ingredient to effect its biological function is presumed to be dependent on release of the native ingredient by resident esterase activities in the skin.

Another type of potentially desirable active ingredient would be a novel combination of one or more molecules (whether covalent or ionic), at least one of which has some beneficial effect on the skin.

### SUMMARY OF THE DISCLOSED TECHNOLOGY

In certain embodiments, the present technology is directed to a method of synthesizing a salicylic acid derivative, the method comprising the steps of: (a) activating a target molecule having a carboxyl, hydroxyl or amine group in the presence of a solvent to produce an activated target molecule; (b) activating salicylic acid on the carboxyl or hydroxyl group to produce activated salicylic acid; and (c) combining the activated target molecule and the activated salicylic acid to produce a salicylic acid derivative.

In other embodiments, the present technology is directed to a method of synthesizing a salicylic acid derivative, the method comprising the steps of: (a) combining a target molecule and salicylic acid with a solvent to provide a solution comprising the target molecule and salicylic acid; and (b) contacting the solution with an activator to provide a solution comprising activated salicylic acid.

In certain embodiments, the present technology is directed to a method of synthesizing a mixture of mono-, di- and poly-substituted salicylates, the method comprising the steps of: (a) combining salicylic acid with target molecule in one or more solvents; and (b) increasing the ratio of di- and poly-substituted salicylates by increasing the molar ratio of salicylic acid to target molecule.

In certain embodiments, the present technology is directed to a mixture of salicylic acid derivatives, the mixture comprising: (a) mono-substituted salicylic acid derivatives; and (b) di- and poly-substituted salicylic acid derivatives.

In certain embodiments, the present technology is directed to a method of synthesizing a mixture of mono-, di- and poly-substituted salicylic acid derivatives, the method comprising the steps of: (a) combining activated salicylic acid with activated target molecule in one or more solvents; and (b) controlling the ratio of di- and poly-substituted salicylic acid derivatives by varying the molar ratio of activated salicylic acid to activated target molecule.

In certain embodiments, the present technology is directed to a method of synthesizing polysalicylic acid, the method comprising activating salicylic acid in the presence of a solvent to produce a polysalicylic acid.

In certain embodiments, the present technology is directed to a pharmaceutical or personal care composition comprising a salicylic acid derivative comprising two or more salicylate functional groups, or a polysalicylate.

In other embodiments, the present technology is directed to a method of inducing the production of a pro-resolvin mediator in a cell or tissue of a patient, the method comprising administering a composition herein; or a method of stimulating the production of a resolvin, the method comprising contacting a cell or tissue of a patient with a composition herein.

### BRIEF DESCRIPTION OF THE DRAWINGS

FIG. 1 shows quantitative levels of 14-HDOHE in peripheral blood mononuclear cells (PBMC) that have been contacted with compositions in accordance with certain embodiments herein.

FIG. 2 shows quantitative levels of 12-HETE in peripheral blood mononuclear cells (PBMC) that have been contacted with compositions in accordance with certain embodiments herein.

FIGS. 3 and 4 show pathways leading to pro-inflammatory and pro-resolving lipid mediators.

FIG. 5 shows a chromatogram with results on the analysis of a composition in accordance with certain embodiments herein, comprising an SA derivative (in this case, a polysalicylic acid) formulated in accordance with certain embodiments herein.

### DETAILED DESCRIPTION

In certain embodiments herein, the present technology is directed to methods for covalently coupling SA to itself to form polymers of various lengths, or of coupling SA to any other target molecule with which it can form a bond. In various embodiments, such bond can be, but not limited to: an ester bond at an available hydroxyl group or an amide bond at an available primary amine. Thus, many new molecular entities can be formed that would be useful for delivering SA bound to other molecules that might confer an additional benefit.

As used herein, the term “salicylic acid derivative” or “SA derivative” refers to a molecule of any compound that is linked or attached with one or more SA moieties (that is, n=1 or more); or a molecule of SA that is linked or attached with any compound that has been combined with the SA (also n=1 or more). As used herein, “linked” or “attached” refer to bonding covalently, ionically or otherwise associated chemically, and also includes bonding through processes such as Fisher esterification and other processes involving bonding with acid at high temperatures. In certain embodiments, the methods discussed herein refer to methods of combining SA with a molecule on which it is desired that the SA be linked or attached (referred to herein as a “target molecule”) to yield the salicylic acid derivative.

As used herein, the term “polysalicylic acid,” “polysalicylate” and “polysalicylic acid molecule” refers to a molecule comprising two or more SA units coupled to each other, and can include straight chains or cyclical structures.

In certain embodiments, the present technology is directed to methods that comprise dissolving SA in an appropriate solvent and activating any active group thereon as a step to forming either the polysalicylic acid or the salicylic acid derivative. For example, one or more of the carboxyl or hydroxyl group may be activated on the SA. Examples of useful SA activators include, but are not limited to the following:

(1) Carbodiimides, including but not limited to: N,N′-Dicyclohexylcarbodiimide (DCC); N,N′-diisopropylcarbodiimide (DIC); N-Cyclohexyl-N′-(2-morpholinoethyl)carbodiimide metho-p-toluenesulfonate (CMC); 1-tert-Butyl-3-ethylcarbodiimide; 1-Ethyl-3-(3-dimethylaminopropyl)carbodiimide (EDC); N,N′-Di-tert-butylcarbodiimide; N-(3-Dimethylaminopropyl)-N′-ethylcarbodiimide; or 1,3-Di-p-tolylcarbodiimide;

(2) Diimidazoles, including but not limited to: 1,1′-Carbonyldiimidazole; 1,1′-Thiocarbonyldiimidazole; or 1,1′-Oxalyldiimidazole;

(3) Uronium and Phosphonium reagents, including but not limited to: O-(Benzotriazol-1-yl)-N,N,N′,N′-tetramethyluronium tetrafluoroborate; (7-Azabenzotriazol-1-yloxy)tripyrrolidinophosphonium hexafluorophosphate; (Benzotriazol-1-yloxy)tris(dimethylamino)phosphonium hexafluorophosphate; N,N,N′,N′-Tetramethyl-O-(N-succinimidyl)uronium tetrafluoroborate (TSTU); 1-[Bis(dimethylamino)methylene]-1H-1,2,3-triazolo[4,5-b]pyridinium 3-oxid hexafluorophosphate (HATU); N,N,N′,N′-Tetramethyl-O-(1H-benzotriazol-1-yl)uronium hexafluorophosphate (HBTU); or (1-Cyano-2-ethoxy-2-oxoethylidenaminooxy)dimethylaminomorpholinocarbenium hexafluorophosphate (COMU).

The embodiments herein contemplate the use of one or more solvents during activation of the target molecule, the SA or both. In certain embodiments, any of the one or more solvents may be the same or different—for example, when referring to more than one solvent, or to a “first” and a “second” solvent, the first and second solvent may, in various embodiments, be the same or different solvents. Examples of useful solvents for dissolving or activating SA, or for dissolving or activating another target molecule with which SA (whether activated or not) will react, include, but are not limited to the following: Polar aprotic solvents, e.g., acetonitrile; dimethylsulfoxide (DMSO); Hexamethylphosphoramide (HMPA); 1,3-Dimethyl-3,4,5,6-tetrahydro-2(1H)-pyrimidinone (DMPU); 1,3-Dimethyl-2-imidazolidinone (DMI); dimethylformamide (DMF); or 1-Methyl-2-pyrrolidinone (NMP).

In certain embodiments, activation of a carboxyl or hydroxyl group of SA using one of the activators above allows it to react to form an ester bond with another molecule of SA, or with any other target molecule that possesses one or more available carboxyl or hydroxyl groups. In certain embodiments, activation of a carboxyl or hydroxyl group of SA using one of the activators above also allows it to react to form an amide bond with a target molecule that possesses an available amine group, such as a primary amine group. To be clear, in some embodiments, an ester bond may be formed by activating one or both of a carboxyl and hydroxyl group. For certain embodiments directed to polysalicylic acids, an ester bond may be formed by activation of a carboxyl group alone, or both a carboxyl and hydroxyl group. For certain embodiments directed to SA derivatives, an ester bond (also referred to herein as a “linkage” can be formed by any of the following methods: activating a carboxyl group on the SA and a hydroxyl group on the target molecule; activating a hydroxyl group on the SA and a carboxyl group on the target molecule; or both can be activated on both the SA and the target molecule.

In the case of reaction of activated SA to form an ester bond with a target molecule possessing an available carboxyl or hydroxyl group, in certain embodiments the reaction can be facilitated by first dissolving the target molecule in an appropriate solvent and activating the available carboxyl or hydroxyl group. Examples of useful carboxyl or hydroxyl group activators include, but are not limited to the following: 1,8-Diazabicycloundec-7-ene (DBU); 1,5-Diazabicyclo[4.3.0]non-5-ene (DBN); Triethylamine (TEA); 2,6-Di-tert-butylpyridine; Phosphazene bases (t-Bu-P4, BEMP); Hünig's base (diisopropylethylamine, DIPEA); or 2,2,6,6-Tetramethylpiperidine (TMP).

In certain embodiments, the activation of SA can be carried out separately from the activation, if any, of a target molecule that contains an available hydroxyl group. As activation of SA is an exothermic process, the reaction flask in which the activation is performed can be cooled in a bath over a period of, in various embodiments, about 1 to about 90 minutes, about 5 to about 60 minutes, or about 1 day to about 3 days or more depending on reaction scale and rate of addition of components. Higher temperatures are generally expected to lead to a faster reaction; in various embodiments, the temperature of the reaction can be about 40 to about 45 degrees Celsius, up to about 80 degrees Celsius, or at room temperature (about 20 to about 25 degrees Celsius). In certain embodiments, one or more carbodiimides may be used for activation of SA, alone or in combination with one or more other molecules such as N-hydroxysuccinimide (NHS). After activation, the coupling reaction can be started by mixing the solution of activated SA with the solution of the target molecule, activated or not, in, for example, a ratio of about 1.5 to about 1 of the two solutions or any other ratio based on desired outcome. In certain embodiments, the presence of excess activator or excess SA will lead to greater amounts of di- and poly-substituted salicylates. The resulting mixture may be stirred at room temperature for, in various embodiments, about 1 hour to about 72 hours, or about 1, about 2, or about 3 days.

When a solution of activated SA alone is allowed to react, polysalicylate will be formed. The extent of polymerization will depend on both reaction time and the concentration of the activated SA, as well as the temperature. When activated SA is reacted with a target molecule other than itself, which may contain one or more available carboxyl or hydroxyl groups, activated or not, and/or one or more amine groups, polysalicylate may be formed in addition to salicylate esters and/or amides of the second molecule. In certain embodiments, the amounts and ratios of the end products may be controlled by varying the relative concentration of SA to one or more of the target molecules, and can thus be engineered or designed to be a mixture of one or more conjugated species. The reaction products can thus be a complex mixture.

In the case where the target molecule contains just one available carboxyl, hydroxyl or amine group, the mixture would in general be expected to contain some unmodified target molecule in addition to a target molecule derivatized with polysalicylate chains of varying length, the range being from one (a single salicylate residue) up to several. If the target molecule contains two or more available carboxyl, hydroxyl or amine groups, or a combination of one or more of both types of available groups, the reaction products would be expected to be a correspondingly complex mixture. In this case, each of the available carboxyl, hydroxyl or amine groups could be derivatized with polysalicylate of chain length varying from zero (no salicylate group) up to several. In certain embodiments, a resultant composition may comprise molecules that have as many as about 2 to about 20 molecules, about 5 to about 20 molecules, about 8 to about 15 molecules about 12 to about 15 molecules, or 20 molecules or more, strung together. In certain embodiments, the average degree of derivatization of the target molecule with salicylate can be varied by changing the molar ratio of activated salicylate to target molecule.

In certain embodiments, the compositions herein can be synthesized using solid state synthesis techniques—for example, carbodiamide activators bound to silica or other insoluble materials.

In certain embodiments, for example, in which activated SA is reacted with activated resveratrol as the target molecule, the differently substituted compounds can generally be separated in a predictable manner. Procedures for the generation, isolation, purification and concentration of SA and SA conjugated target molecules have been developed; these procedures allow separation and isolation of specifically substituted end products (for example, resveratrol salicylates). Similar methods can be applied to resolve the components of mixtures created by reaction of activated SA with target compounds other than resveratrol that contain one or more available hydroxyl or primary amine groups.

In certain embodiments, the methods herein further comprise the step of drying the resultant solution of a reaction herein to produce a solid salicylic acid derivative. In other embodiments, the final product need not be solid but may comprise any of the following, in whole or part: a liquid, a gel, a sol, a suspension, a foam, a colloid, an aerosol, an emulsion, a spray or a fluid.

Reaction products created using the technology described in this application can be included as ingredients in any of various types of personal care (including cosmetic), pharmaceutical (including veterinary) formulations, including but not limited to: creams, blush, lotions, serums, foundation makeup, powders, eyeshadow, eyeliner, mascara, lip color (e.g., lipstick and lipbalm), ointments, salves, unguents, balms, gels, oils, mulls, foams, masks, soaps, body washes, shampoos, hair conditioners, sunscreen, astringents, exfoliating agents, deodorants, treatments for acne, pimples, warts, eczema, rosacea; fungal treatments, nail treatments and colorants, sunburn treatments, facial peels and the like.

As described herein, in certain embodiments the target molecule for reaction with activated SA is any molecule of interest containing a carboxyl, hydroxyl or amine group. Useful target molecules can include, but are not limited to any of the following: Exemplary carboxylic acids that can be used to form ester of Salicylic Acid include, but are not limited to:

Substituted or unsubstituted saturated monocarboxylic acids, such as acetic acid, propionic acid, butyric acid (C4), valeric acid, hexanoic acid, caprylic acid (C8), lauric acid, stearic acid (C18), isostearic acid (branched C18), linoleic acid, linolenic acid, myristic acid (C14), arachidic acid (C20), arachidonic acid, erucic acid, behenic acid (C22), lauric acid (C12), capric acid (C10), caproic (C6), and palmitic acid (C16); unsaturated monocarboxylic acids, such as acrylic acid, methacrylic acid, sorbic acid, oleic acid, linoleic acid, linolenic acid, docosahexaenoic acid, and eicosapentaenoic acid or any such acid C2 to C25 or greater;

Amino acids, such as arginine, glutamine, and tyrosine;

Keto acids, such as pyruvic acid and acetoacetic acid;

Aromatic carboxylic acids, such as ascorbic acid, benzoic acid, salicylic acid, 2 and 3 furoic acid and ferulic acid; di- and tri-carboxylic acids, such as oxalic acid, malonic acid, malic acid, succinic acid, and glutaric acid.

Also suitable are carboxylic acids substituted with cyclic disulfide groups, e.g., lipoic acid. The designation “C” followed by a number indicates the number of carbon atoms in the alkyl chain.

In various embodiments, it may be desirable to include one or more botanical extracts in the compositions. If so, suggested ranges are about 0.0001 to about 10%, about 0.0005 to about 8% or about 0.001 to about 5% by weight of the total composition. Suitable botanical extracts include extracts from plants (herbs, roots, flowers, fruits, vegetables, seeds, leaves, pollen, nectar) ; for example, yeast ferment extract, padica pavonica extract, thermus thermophilis ferment extract, camelina sativa seed oil, boswellia serrata extract, olive extract, aribodopsis thaliana extract, acacia dealbata extract, acer saccharinum (sugar maple), acidophilus, acorns, aesculus, agaricus, agave, agrimonia, algae, aloe, citrus, brassica, cinnamon, orange, apple, blueberry, cranberry, peach, pear, lemon, lime, pea, seaweed, caffeine, green tea, chamomile, willowbark, mulberry, poppy, and any other type of botanical extract Further examples include, but are not limited to, Glycyrrhiza Glabra, Salix Nigra, Macrocycstis Pyrifera, Pyrus Malus, Saxifraga Sarmentosa, Vilis Vinifera, Moms Nigra, Scutellaria Baicalensis, Anthemis Nobilis, Salvia Sclarea, Rosmarinus Officianalis, Citrus Medica Limonum, Panax Ginseng, and mixtures thereof.

Other useful target molecules include any of the following:

Sugars—including mono saccharides such as glucose, ribose, fructose, mannose, galactose; disaccharides such as sucrose, trehalose, maltose, cellobiose; polysaccharides such as gums, chitan; amino sugars and amino sugar derivatives such as Hyaluronic Acid and Chondroitin sulphates;

Lipids—including phospholipids, sterol lipids, sphingolipids, free fatty acids, ceramides and cholesterols;

Polyphenolics—including tannins, ellagitanins, resorcinol, and other polyphenols,

Organic Acids—including gallic acid, ursolic acid, hydrocinnamic acids such as ferulic acid, and heterocyclic carboxylic acid such as furoic acids;

Antioxidants—including ascorbic acid, Nordihydroguaiaretic acid (NDGA);

Flavones and flavonoids—including Quercetin, anthrocyanidins, hesperidin;

Vitamins—including A, B, C, D, E and K;

Amino acids—including Histidine, Alanine, Isoleucine, Arginine, Leucine, Asparagine, Lysine, Aspartic acid, Methionine, Cysteine, Phenylalanine, Glutamic acid, Threonine, Glutamine, Tryptophan, Glycine, Valine, Ornithine, Proline, Selenocysteine, Serine, Tyrosine;

Xanthines—including caffeine, theophylline and theobromine;

Mycosporine-like amino acids;

Enzymes—including glutathione S-transferases (GST's), superoxide dismutases (SOD), peroxidases, catalase; and

Organic polymers—including dextran, polyglucosamine, polysaccharides, polyacrylates, polyvinylpyrrolidone.

In certain embodiments, the methods herein comprise the following steps: (1) obtaining the target molecule, subjecting it to an activator to produce activated target molecule; (2) obtaining salicylic acid, subjecting it to an activator to produce activated salicylate; and (3) combining (1) and (2) to produce the SA derivative.

In certain embodiments, the activation can be carried out separately for the target molecule and salicylic acid. In one non-limiting example, the target molecule can be activated by treatment with activator in a solvent. Similarly, salicylic acid can be activated by treatment with a solvent using an excess of a salicylic activator. In various embodiments, the coupling reaction may then be run in a mixture of the two solvents. In other examples, carbodiimides may be used for activation of salicylic acid, alone or in combination with other molecules such as N-hydroxysuccinimide (NHS).

In a non-limiting example, the activation of the target molecule can be run for about 10 min at room temperature. As salicylic acid activation is an exothermic process, the reaction flask may, in certain embodiments, be kept in a cooling bath while the salicylic acid activator is added in portions over a period of about 1 to about 60 minutes, or about 5 to about 30 minutes, or about 10 to about 20 minutes, depending on the reaction scale and temperature. In certain embodiments, upon completion of salicylic acid activator addition, the cooling bath may (but need not) be removed. In various embodiments, this activation can also be run for about 30 to about 90 minutes, or more, including upwards of 3 to 5 days. The reaction mixture containing activated target molecule may then be combined with the activated salicylic acid. The resulting mix may be stirred at room temperature for, in various embodiments, up to about 72 hours, or about 1, about 2, or about 3 days.

In certain embodiments, the polysalicylates product distribution is dependent on the molar ratio of activated salicylic acid to activated target molecule, with higher ratio favoring a more substituted product. Thus, in certain embodiments, the SA derivative mixture comprises mono-, di- and poly-substituted SA derivatives; the ratios of the various substituted SA derivatives in the mixture can be controlled by varying the molar ratio of activated salicylic acid to activated target molecule, with higher ratios of the activated salicylic acid generally leading to more highly-substituted products. Thus in certain embodiments, the SA derivative mixture comprises mono-, di- and poly-substituted target molecule, and other polymeric salicylates on one or more hydroxyl groups of the target molecule—in other words, one or more salicylic acid molecules conjugated to any one or more/all hydroxyls on the target molecule.

Thus, in certain embodiments, an investigator can optimize the relative proportions of mono-, di- and poly-salicylate-substituted target molecule by varying the molar ratios of the activated salicylic acid and activated target molecule. For example, the investigator may choose to increase the amount of mono-substituted SA derivatives by decreasing the molar ratio of activated salicylic acid to activated target molecule; conversely, if he wishes for a greater proportion of di- or polysalicylate-substituted target molecule (for example, n=12 to 20 or greater), he can optimize these amounts by increasing the molar ratio of activated salicylic acid to activated target molecule. Indeed, in certain embodiments, the investigator may decide that maximizing the proportion of higher-substituted end product is a more beneficial way to engage the methods herein.

In certain embodiments, the present technology is directed to a method of synthesizing a mixture of mono-, di- and poly-substituted SA derivatives, the method comprising the steps of: (a) combining activated salicylic acid with activated target molecule in one or more solvents to produce one or more SA derivatives; and (b) controlling the ratio of di- and poly-substituted SA derivatives by varying the molar ratio of activated salicylic acid to activated target molecule. Certain embodiments of the present technology include the step of controlling the hydrolysis of the poly-substituted product to yield a desired distribution of mono-, di- and poly-salicylate-modified derivative.

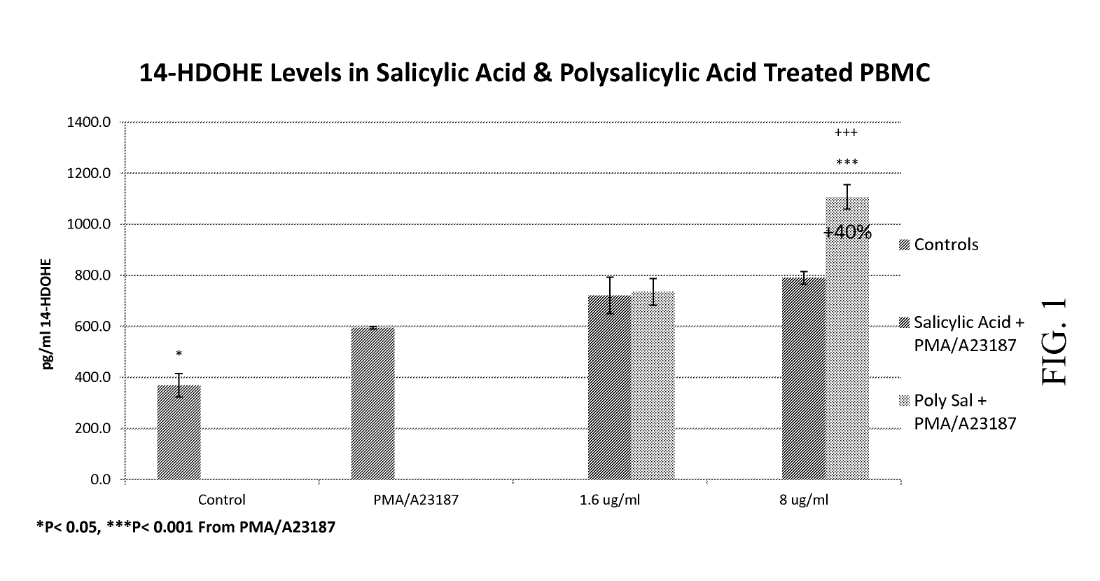

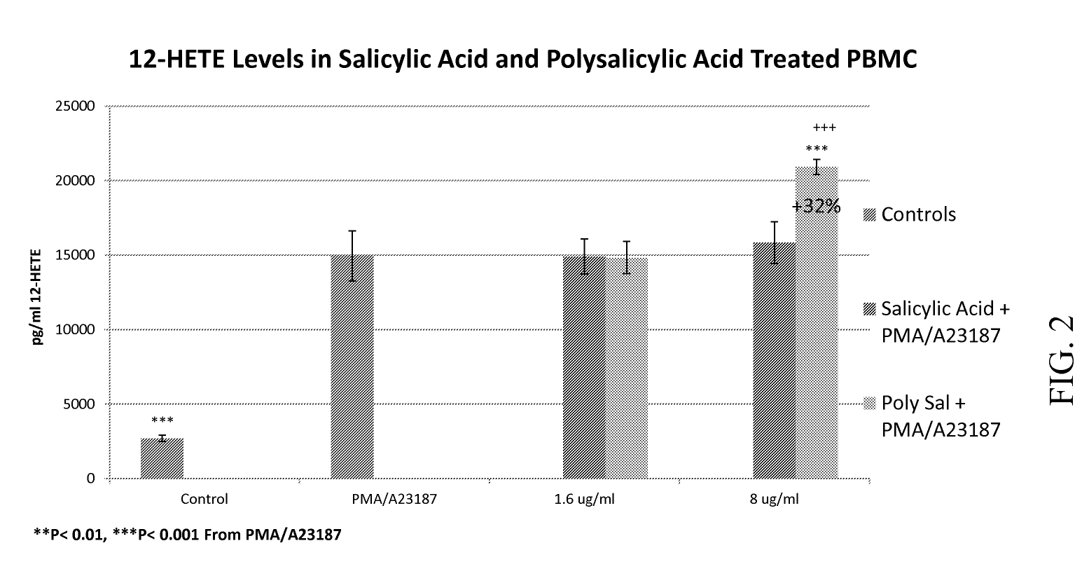

In certain embodiments herein, the resultant molecules exhibit superior action on living cells by, for example, stimulating production of resolvins. Resolvins are pro-resolving (anti-inflammatory) lipid mediators, and are compounds that are made by the human body from the omega-3 fatty acids eicosapentaenoic acid (EPA) and docosahexaenoic acid (DHA), as well as arachidonic acid (AA). The resolvins comprise: E-series resolvins, D-series resolvins, lipoxins, protectins and maresins, as well as Annexin Al and hydrogen sulfide. Substantial resolvin production occurs via the COX-2 pathway, especially in the presence of aspirin. Experimental evidence indicates that resolvins reduce cellular inflammation by inhibiting the production and transportation of inflammatory cells and chemicals to the sites of inflammation. In certain embodiments, the compositions produced herein stimulate resolvins or resolvin precursors as seen, for example, in FIGS. 1 and 2.

12-Hydroxyeicosatetraenoic acid (12-HETE) is a derivative of the 20 carbon polyunsaturated fatty acid, arachidonic acid. The presence of 12-HETE indicates that 12-LOX enzymes are present and active. 12-LOX enzymes depend on ALOX12, ALOX12B, ALOXE3 genes. Thus, an increase of 12-HETE production in cells can lead to the conclusion that 12-LOX enzymes are present and modulated by the compound that has been contacted with the cells.

Moreover, activation of the 12-LOX enzymes is necessary for the production of 14-HDOHE, a precursor of the resolvin Maresinl. 12-LOX is also involved in the synthesis of two other resolvins, LXA4/LXB4. Thus, in certain embodiments, quantifying 12-HETE or 14-HDOHE can lead to a conclusion that the 12-LOX pathway is active and that Maresinl, LXA4 or LXB4 are potentially produced.

Additionally the 12-HETE pathway is involved in the trioxilin and hepoxilin pathways which can be of interest in psoriasis.

FIG. 1 shows results of 14-HDOHE levels in peripheral blood mononuclear cells (PBMC) that have been treated with: (1) nothing (control); (2) PMA plus A23187 (a phorbol ester and a calcium ionophore to trigger an inflammatory response); (3) PMA plus A23187 in the presence of salicylic acid; and (4) PMA plus A23187 in the presence of polysalicylic acid. The leftmost bar shows the 14-HDOHE levels produced by the control cells. The next bar shows the levels with PMA plus A23187 treatment. The next two sets of adjacent bars show a comparison of the effects of treatment with PMA plus A23187 in the presence of salicylic acid (on left of each group) and polysalicylic acid (on right of each group) in amounts of 1.6 micrograms per milliliter (μg/mL) and 8 micrograms per milliliter (μg/mL), respectively.

FIG. 2 shows results of 12-HETE levels in PBMC that have been treated with: (1) nothing (control); (2) PMA plus A23187 (a phorbol ester and a calcium ionophore to trigger an inflammatory response); (3) PMA plus A23187 in the presence of salicylic acid; and (4) PMA plus A23187 in the presence of polysalicylic acid. The leftmost bar shows the 12-HETE levels produced by the control cells. The next bar shows the levels with PMA plus A23187 treatment. The next two sets of adjacent bars show a comparison of the effects of treatment with PMA plus A23187 in the presence of salicylic acid (on left of each group) and polysalicylic acid (on right of each group) in amounts of 1.6 micrograms per milliliter (μg/mL) and 8 micrograms per milliliter (μg/mL), respectively.

Thus, in various embodiments, the compositions herein, when contacted with cells or tissues, result in an increase in the levels of pro-resolvin mediators such as 12-HETE or 14-HDOHE, in amounts of: at least about 25%, at least about 30%, about 20 to about 75%, about 25 to about 65%, about 30 to about 55%, about 30 to about 50% or about 30 to about 40% when compared to levels measured when contacted with salicylic acid alone. As can be seen, for example, in FIGS. 1 and 2, while the addition of salicylic acid to the cells or tissues can result in a noticeable increase over the control, the addition of a salicylic acid derivative such as polysalicylic acid can result in a larger and statistically significant increase. Thus, an unexpectedly beneficial effect is seen with compositions having PSA molecules, to an extent that would not be predicted based on mere increase of the number of individual SA molecules. That is, the increase with the addition of salicylic acid derivatives such as PSA is greater than would be expected based on the sum of activities of the individual SA molecules in the PSA, if they were not linked together.

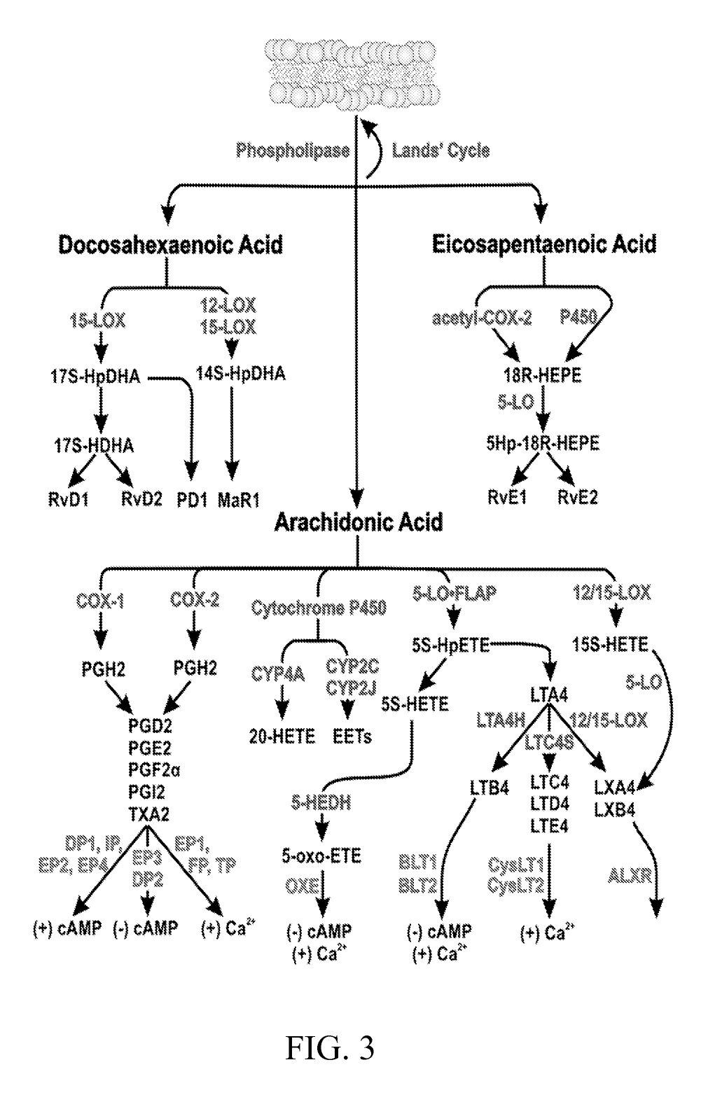

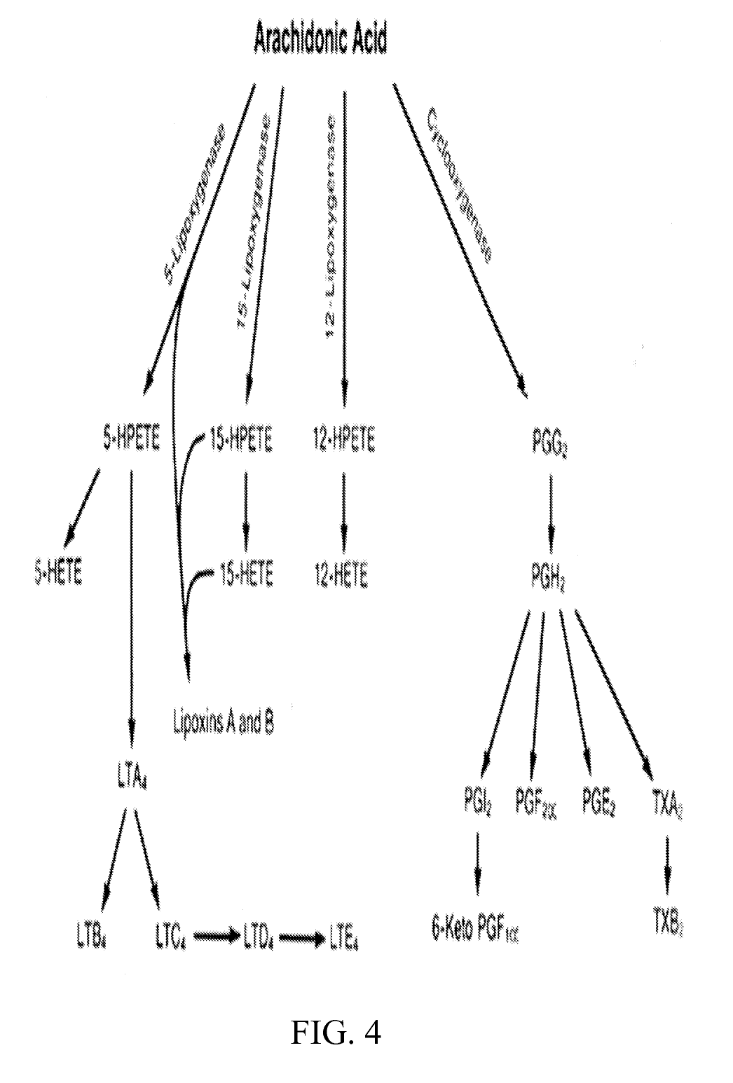

Certain pathways lead to the metabolites of interest for certain embodiments herein—for example, a pathway from DHA leads to 14-HDHA (which is the same as 14-HDOHE) which leads to Maresinl, a desirable pro-resolving mediator. FIGS. 3 and 4 show other certain pathways leading to the metabolites of interest for certain embodiments herein—specifically, the pathways that lead from DHA to Maresinl and from AA to the lipoxins (e.g., LXA4 and LXB4) (FIGS. 3); and 12-HETE, which shows 12 lipoxygenase activity (FIG. 4)

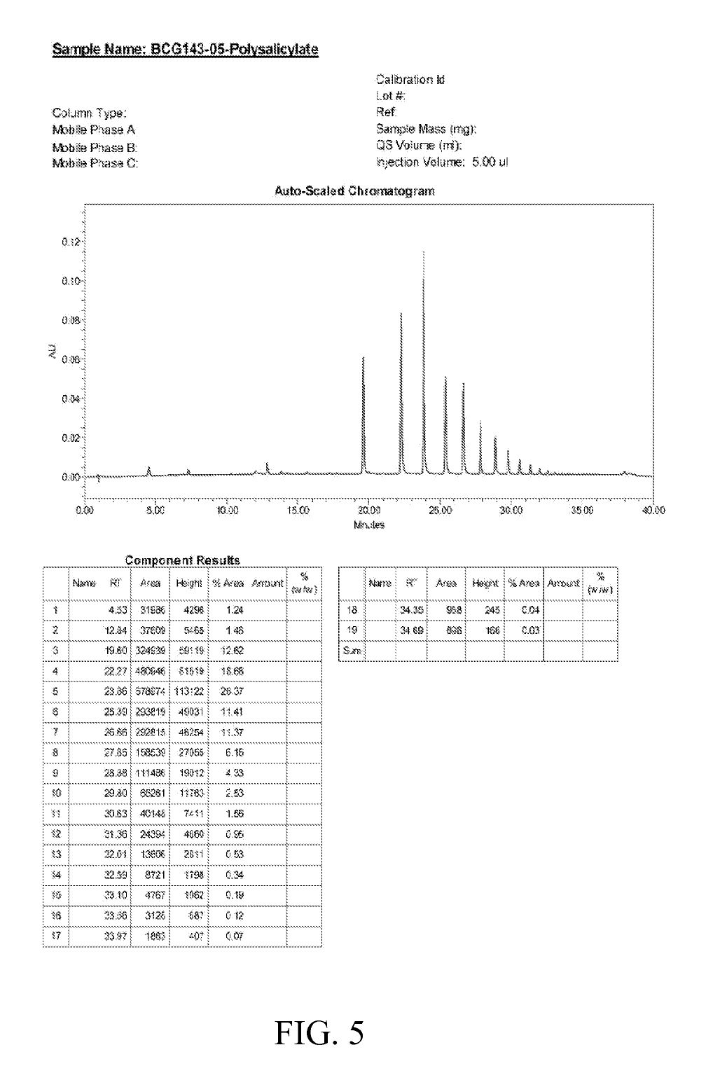

FIG. 5 shows an autoscaled chromatogram with results on the analysis of a composition in accordance with certain embodiments herein, comprising an SA derivative (in this case, a polysalicylic acid) formulated in accordance with certain embodiments herein. As can be seen, going from left to right, each peak represents an added salicylic acid molecule—that is, mono, di, tri, and so on. As can be seen, a desirable distribution of the groups can be achieved using the methods herein.

### EXAMPLE

The methods discussed herein were used to generate molecules, of which many were tested to determine their properties. In particular, a sample of polysalicylic acid was created, which comprised various distributions of mono-, di-, tri and poly-salicylic acid, for example, as shown in FIG. 5.

The resultant SA derivative was evaluated at different doses on the synthesis of 8 intermediate lipids of the resolution pathways in PMBC with PMA/A23187 stimulation.

PMBC cells were seeded and allowed to settle for 1 hour; the test compounds were then added and allowed to incubate for an additional hour. The inflammatory stimulus (PMA plus A23187) was added for one hour, and supernatants were collected and analyzed for lipid intermediates.

The results of the analysis showed the following: the compositions herein comprising polysalicylic acid showed definite anti-inflammatory effects (decrease of PGE2 and LTB4 secretion) and increased 14-HDOHE and 12-HETE production—two precursors of D-Resolvins, Maresins and Protectins.

In addition, the polysalicylic acid compounds herein were shown to increase 12-HETE and 14-HDOHE without completely blocking 15-HETE secretion, thus allowing low activation of the 5-lipoxygenase pathway, which is necessary for production of lipoxins and the E-series resolvins. Thus, it showed high potential for promoting synthesis of the entire spectrum of specialized pro-resolving mediators (SPMs). Induction of pro-resolving mediators is conducive to an anti-inflammatory effect—that is, it is a valuable step toward reducing inflammation, a hallmark of many diseases.

In summary, the present technology is highly superior for many reasons, including but not limited to the following: the methods here are different from those known in the art; the methods here generate salicylates as products, and these products can be confirmed (presence and identity) by both spectral and chemical analysis; and the methods here can be used to obtain many different salicylates, including polysalicylates. The synthetic approaches and subsequent purification methods used herein can avoid toxic and mutagenic solvents, and are highly amenable to scaleup.

Other advantages include the following: the methods herein can provide neutral forms of SA that are highly stable and permit sustained delivery and timed release of active ingredients. For example, in certain embodiments, the present technology is directed to methods of sustained time release of salicylic acid via enzymes in the skin, e.g., skin esterases. The skin can derive monosalicylic acid from di- and polysalicylic acids; as such, compositions of the present technology can have desirable benefits for skin.

In other embodiments, the present technology is directed to methods of increasing target molecule stability through the methods herein. For example, in certain embodiments, the methods and compositions herein may be used to stabilize unstable molecules such as, e.g., NDGA or resveratrol.

In certain embodiments, the methods and compositions herein can provide enhancement of skin penetration. Options include formulations in anhydrous or hydrophilic systems.

It will be appreciated by those skilled in the art that the methods and compositions disclosed in accordance with the present technology can be used in connection with a wide variety of methods and materials. Although the technology has been disclosed in the context of certain exemplary embodiments, those skilled in the art will readily appreciate that the present technology extends beyond the specifically disclosed embodiments to other alternative embodiments. Thus, it is intended that the scope of the invention should not be limited by the particular disclosed embodiments described above.

What is claimed:

1. A method of synthesizing a salicylic acid derivative, the method comprising the steps of:
(a) combining a target molecule and salicylic acid with a solvent to provide a solution comprising target molecule and salicylic acid; and
(b) contacting the solution with an activator to provide a solution comprising activated salicylic acid.

2. The method of claim 1, further comprising the step of:
(c) drying the solution to produce a solid salicylic acid derivative.

3. The method of claim 1, wherein the contacting step of (b) produces a mixture of salicylic acid derivatives.

4. The method of claim 3, wherein the mixture of salicylic acid derivatives comprises mono-, di- and poly-substituted salicylates on one or more hydroxyl, carboxyl or amine group of the target molecule.

5. The method of claim 1, wherein the solvent is acetonitrile; dimethylsulfoxide (DMSO); Hexamethylphosphoramide (HMPA); 1,3-Dimethyl-3,4,5,6-tetrahydro-2(1H)-pyrimidinone (DMPU); 1,3-Dimethyl-2-imidazolidinone (DMI); dimethylformamide (DMF); or 1-Methyl-2-pyrrolidinone (NMP) or dimethylaminopyridine (DMAP).

6. The method of claim 1, wherein the activator is chosen from the following:
(a) a carbodiimide chosen from N,N′-Dicyclohexylcarbodiimide (DCC); N,N′-diisopropylcarbodiimide (DIC); N-Cyclohexyl-N′-(2-morpholinoethyl)carbodiimide metho-p-toluenesulfonate (CMC); 1-tert-Butyl-3-ethylcarbodiimide; 1-Ethyl-3-(3-dimethylaminopropyl)carbodiimide (EDC); N,N′-Di-tert-butylcarbodiimide; N-(3-Dimethylaminopropyl)-N′-ethylcarbodiimide; or 1,3-Di-p-tolylcarbodiimide;
(b) a diimidazole chosen from 1,1′-Carbonyldiimidazole; 1,1′-Thiocarbonyldiimidazole; or 1,1′-Oxalyldiimidazole; or
(c) a uronium or phosphonium reagent, including but not limited to: O-(Benzotriazol-1-yl)-N,N,N′,N′-tetramethyluronium tetrafluoroborate; (7-Azabenzotriazol-1-yloxy)tripyrrolidinophosphonium hexafluorophosphate; (Benzotriazol-1-yloxy)tris(dimethylamino)phosphonium hexafluorophosphate; N,N,N′,N′-Tetramethyl-O-(N-succinimidyl)uronium tetrafluoroborate (TSTU); 1-[Bis(dimethylamino)methylene]-1H-1,2,3-triazolo[4,5-b]pyridinium 3-oxid hexafluorophosphate (HATU); N,N,N′,N′-Tetramethyl-O-(1H-benzotriazol-1-yl)uronium hexafluorophosphate (HBTU); or (1-Cyano-2-ethoxy-2-oxoethylidenaminooxy)dimethylaminomorpholinocarbenium hexafluorophosphate (COMU).

7. The method of claim 6, wherein the salicylic acid is activated by treatment with a carbodiimide of (a) in combination with N-hydroxysuccinimide (NHS).

8. The method of claim 1, wherein the target molecule is salicylic acid.

9. A method of synthesizing a mixture of mono-, di- and poly-substituted salicylates, the method comprising the steps of:
(a) combining salicylic acid with a target molecule in one or more solvents; and
(b) increasing the ratio of di- and poly-substituted salicylates by increasing the molar ratio of salicylic acid to target molecule.

10. A mixture of salicylic acid derivatives, the mixture comprising:
(a) mono-substituted salicylic acid derivatives; and
(b) di- and poly-substituted salicylic acid derivatives.

11. A method of synthesizing a mixture of mono-, di- and poly-substituted salicylic acid derivatives, the method comprising the steps of claim 1.

12. The method of claim 11, further comprising the step of:
(c) controlling the ratio of di- and poly-substituted salicylic acid derivatives by varying the molar ratio of salicylic acid to target molecule.

13. The method of claim 12, further comprising the step of controlling the hydrolysis of the poly-substituted salicylic acid derivative to yield a desired distribution of mono-, di- and poly-salicylate-modified salicylic acid derivative.

14. A polysalicylic acid derivative synthesized by the method of claim 1.

15. A method of synthesizing polysalicylic acid, the method comprising activating salicylic acid in the presence of a solvent to produce a polysalicylic acid.

16. A personal care composition comprising a salicylic acid derivative comprising two or more salicylate functional groups, or a polysalicylate.

17. A pharmaceutical composition comprising a salicylic acid derivative comprising two or more salicylate functional groups, or a polysalicylate.

18. A method of inducing the production of a pro-resolvin mediator in a cell or tissue of a patient, the method comprising administering a composition of claim 10 to the patient.

19. The method of claim 18, wherein the pro-resolvin mediator is 12-HETE or 14-HDOHE.

20. A method of stimulating the production of a resolvin, the method comprising contacting a cell or tissue of a patient with a composition of claim 10.

21. The method of claim 20, wherein the resolvin is a maresin, D-series resolvin, E-series resolvin, a lipoxin, a protectin or a combination of any of the foregoing.
